(19) United States
(12) Patent Application Publication
(10) Pub. No.: US 2005/0148945 A1
(43) Pub. Date: Jul. 7, 2005

(54) Medical syringe

(76) Inventor: Yong S. Chen, Glendora, CA (US)

Correspondence Address: Boniard I. Brown, 1500 West Covina Parkway, #113, West Covina, CA, 91790-2793, US

(21) Appl. No.: 10/746,214

(22) Filed: Dec. 24, 2003

(51) Int. Cl. A61M 5/32

(52) U.S. Cl. 604/198

(57) ABSTRACT

An improved medical syringe enables quick operation of a handle member to position a needle and syringe barrel in selected positions to extend and withdraw a needle to provide a needle system or a needle-less system.

### BACKGROUND AND SUMMARY OF THE INVENTION

The syringe of the present invention has a retractable needle positionable by a handle engageable in any of a plurality of stops or notches by simple manual manipulation, whereby the syringe is convertible from a needle to a needle-less system by extending or withdrawing the needle. The needle may be extended for aspirating a solution into the syringe from a bottle or an ampoule. The solution may then be injected into a patient directly or through an intravenous port. The needle may be retracted to expose the blunt tip of the syringe for engaging a needle-less intravenous port.

The syringe of the invention is versatile in other respects. Conversion from a needle system to a needle-less system or vice versa is readily accomplished by manipulating a control handle without touching the needle, thus greatly reducing the risk of accidentally sticking the operator with the needle. Removal and replacement of a needle are eliminated. The conversion from a needle system to a needle-less system and vice versa may be repeated again and again for a single patient in accordance with the patient's needs. A needle may be used repeatedly without removal from the syringe, thus eliminating the discarding of many needles, and the attendant high costs of such discarding. To dispose of the syringe or when the syringe is temporarily out of service, the needle is readily retractable into the syringe housing by operation of the handle. The foregoing advantages and convenience save substantial medical professional time in assembling and filling of syringes. The capability provided by the syringe of the invention can avoid delays and is even life-saving in some circumstances.

In a preferred embodiment, the present invention comprises a syringe barrel slidable in an outer housing, a hollow needle extending forwardly from the syringe barrel and movable with the barrel to penetrate a resilient member and extend outwardly from the housing. A control handle is pivotally mounted and has means to engage any of a plurality of spaced-apart stops or notches for the selective positioning of the needle to extend it beyond the forward end of the housing or to retract the needle within the housing.

### BRIEF DESCRIPTION OF THE DRAWINGS

FIGS. 1, 2 and 3 are sectional views of a preferred embodiment of the present invention, showing the needle and other components in different positions in use in operation of the syringe of the invention;

FIG. 4 is a top view of the syringe of FIGS. 1-3;

FIG. 5 is a sectional view taken at line 5-5 in FIG. 3;

FIG. 5A is a sectional view taken at line 5A-5A in FIG. 4;

FIG. 6 is a perspective view of the syringe of FIGS. 1-3 with its needle extended;

FIGS. 7, 8 and 9 are sectional views of a second embodiment of the syringe of the invention, showing components in successive positions of operation;

FIG. 10 is a top view of the embodiment of the invention shown in FIGS. 7-9;

FIG. 11 is a sectional view taken at line 11-11 in FIG. 9; and

FIG. 12 is a perspective view of the syringe of FIGS. 7-9.

### DESCRIPTION OF THE PREFERRED EMBODIMENTS

Referring to the drawings, a preferred embodiment 10 of the present invention comprises an outer housing 12, a syringe body or barrel 14 of generally cylindrical configuration, and a plunger 16 slidable in the syringe body 14.

A helical spring 18 is disposed between an end wall 20 of the housing and an end wall 24 of the plunger 16. A hollow needle 22 is extensible through end wall 24 of the syringe to communicate with the interior of the syringe.

A resilient seal 26 is threadedly mounted in the end wall 20. The needle 22 extends through the resilient seal member 26 and tubular end member 28. The needle preferably has an inclined sharp end portion to facilitate entry into a plug of a vial or an opened ampoule for aspiration of liquid, or for injection into a person.

Control handle 34 is manually pivotal about a pivot 36 on a bracket secured on a forward portion of syringe barrel 14, as shown. The handle 34 extends outwardly via an upper opening in the housing 12, as shown. As shown in FIGS. 5 and 5A, a spring 38 having a coiled central portion and respective arms engaging the handle and the bracket engages the handle 34 and the bracket mounted on the syringe housing, thus to bias the handle 34 upwardly and outwardly relative to the pivot at 36.

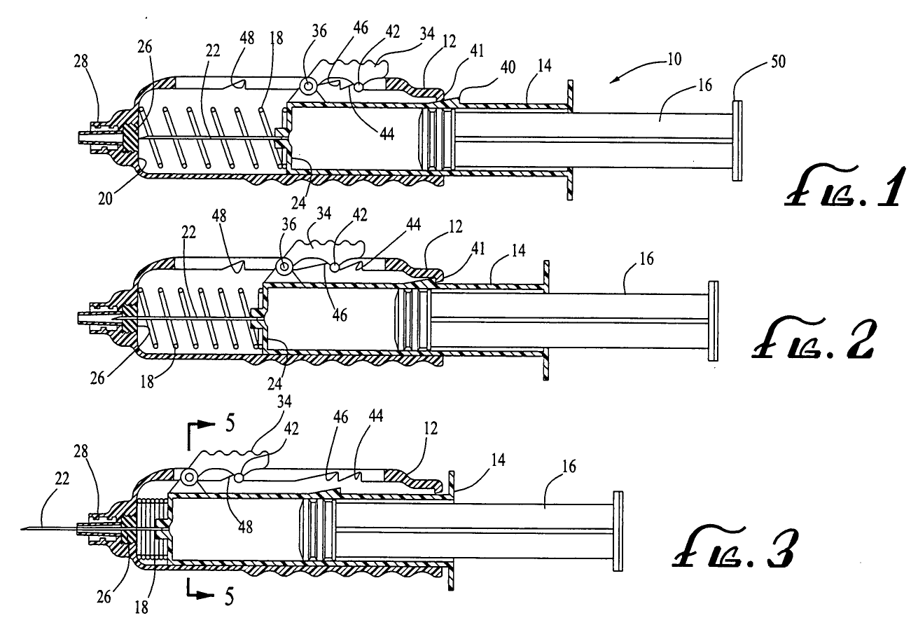

In assembling the syringe of the invention, a foot or ridge 40 on the syringe is first positioned outwardly of housing 12, as shown in FIG. 1. As assembly proceeds, foot 40 moves inside the housing 12, as shown in FIG. 2, and engages a bent end portion 41 to prevent the syringe 14 from moving outwardly on housing 12. The syringe is then movable to extend needle 22 to pierce seal 26, as indicated in FIG. 3. Housing 12 is at the lower portion of undulating contour, as shown, for improved manual gripping.

A rod portion 42 on the handle 34 is adapted to engage in any of the notches 44, 46 or 48 defined in the upper portion of the housing. In utilizing the syringe, portion 42 of the handle is first engaged in the first notch 44. To move the handle portion to engage in the second notch 46, it is only necessary to push the handle a short distance forward to engage the second notch 46 and cause the needle to pierce and extend through the seal member 26. Once the handle is moved into the second notch 46, the syringe cannot then be moved back to notch 44 because the foot 40 is retained by the end edge of the housing 12.

FIG. 3 shows the handle engaged in the third notch 48 so that needle 22 extends outwardly from the end of the housing for use in injection of medication into a person or aspiration of medication or drug from a bottle or an ampoule. In this position, the force of the compressed spring 18 is resisted by the engagement of handle rod portion 42 in the third notch 48. The needle remains in this position until released by manual pressure on handle 34.

In utilizing the syringe, plunger 16 is axially movable outwardly by pulling on outer cap 50 of the plunger to produce suction in the syringe to draw liquid from an ampoule or bottle, etc. Urging the plunger inwardly produces pressure in the syringe as for injecting via the needle 22 or via tubing connected to patient to inject liquid into a patient, bottle or ampoule.

Further, a watertight seal at needle 22 prevents liquid in the syringe from retrograde flow into housing 12 when the tip of the housing is attached to a female connector onto intravenous tubing, regardless of whether positive or negative pressure is applied to the syringe plunger with the syringe plunger pushed forward or pulled backward.

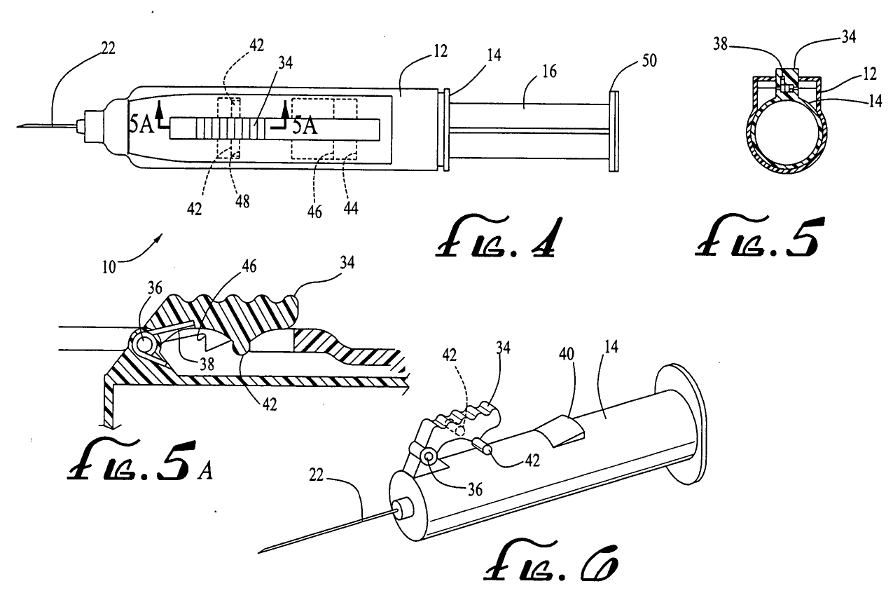

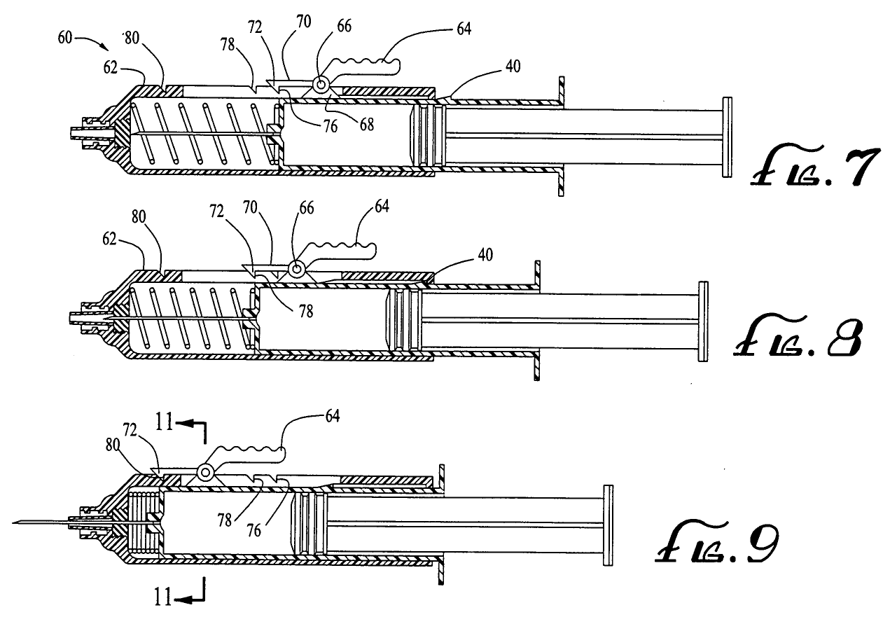

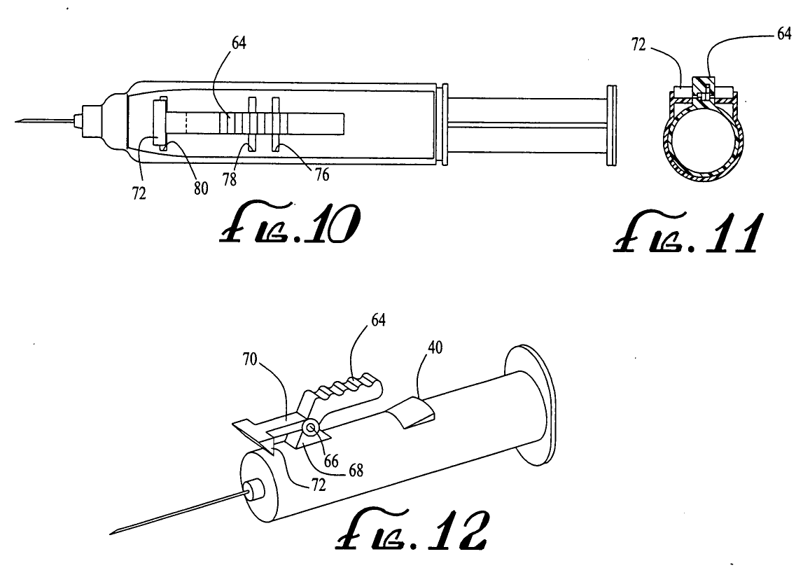

A second embodiment of the present invention is illustrated in FIGS. 7-12. This embodiment comprises certain improvements or alternate features which differ from the embodiment of FIGS. 1-6. Features of this embodiment which are in common with the first embodiment are identified by the same reference numerals.

Like the embodiment of FIGS. 1-6, the device of FIGS. 7-12 may be used as a needle system and also as a needle-less system.

The embodiment of FIGS. 7-12 differs from that of FIGS. 1-6 in that its upper body portion and upper features are flatter and lower and in that notches 76, 78 and 80 are defined differently and differently positioned, as shown. A handle 64 is pivotally mounted relative to the syringe, as in the embodiment of FIGS. 1-6. A hook portion 70 is pivotal by means of handle 64 to engage the hook in a selected notch 76, 78, or 80.

As with the embodiment of FIGS. 1-6, an important feature of the invention is the provision of means whereby simply pushing on the handle 64 converts the syringe from a needle to a needle-less device. With the syringe in its forward position of FIG. 9, with the hook 72 disposed in the third notch 80, the needle extends outwardly from the housing, as shown in FIG. 9. With the needle in its outward position, the user maintains pressure on the spring.

The components remain in steady positions with needle extended until released by manual downward pressure on the handle and pushing forwardly on the handle to release the prong from the third notch 80. The spring of the syringe urges the syringe only as far as the position shown in FIG. 8, wherein the needle is fully retracted into the tip of housing, as shown in FIG. 8.

The prong 72 is stopped at a first position by engagement with the first notch 76 and only needs to be pushed a short distance forwardly to pierce the seal and engage the second notch 78. The prong is released from the notch by pushing forward slightly and pressing downwardly on the handle 64.

It will be understood that various changes and modifications may be made from the preferred embodiments discussed above without departing from the scope of the present invention, which is established by the following claims and equivalents thereof.

1. A medical syringe comprising:
an outer housing having forward and rear end portions,
a syringe barrel of curvilinear cross-section slidable in the outer housing,
a resilient seal in a forward portion of the outer housing,
a hollow needle extending forwardly from the syringe barrel and movable therewith to penetrate the seal member and extend outwardly from the forward end of the outer housing,
spring means between the syringe barrel and a forward wall portion of the housing,
means to apply pressure on liquid in the syringe barrel and to apply partial vacuum thereto for selective movement of the syringe barrel to compress the spring means or to draw liquid into the syringe barrel via said hollow needle,
a plurality of spaced-apart stops defined on an upper portion of the outer housing, and
a control handle pivotal relative to the syringe barrel, said handle having means to engage said stops for selective positioning of the outer housing and said needle thereon to extend the needle beyond the forward end portion of the outer housing, and to retract the needle within the outer housing end portion.

2. A syringe according to claim 1, wherein the outer housing is generally curvilinear in cross-section.

3. A syringe according to claim 1, wherein the control handle is biased outwardly of a pivot on the syringe barrel by spring means engaging the handle and the syringe barrel, the spring having arms engaging the handle and the syringe housing.

4. A syringe according to claim 1, wherein said syringe barrel is of generally cylindrical cross-section.

5. A syringe according to claim 1, wherein said means to apply pressure and partial vacuum comprises a plunger slidable in the syringe barrel.

6. A syringe according to claim 1, wherein the needle is substantially coaxial with the syringe barrel.

7. A syringe according to claim 1, wherein the spring means comprises a coil spring.

8. A syringe according to claim 1, wherein a forward portion of the outer housing is of reduced cylindrical configuration and wherein said seal is mounted.

9. A syringe according to claim 7, wherein said resilient seal member in the forward portion of the outer housing is generally coaxial with the syringe barrel.

10. A syringe according to claim 1, wherein the plurality of stops on the upper housing to receive the handle means comprises spaced-apart notches in the upper wall of the outer housing, and the handle means to engage in the notches is a handle portion adapted to engage in the notches.

11. A syringe according to claim 1, wherein the means to engage the stops comprises a prong on the handle means.

12. A syringe according to claim 10, wherein the means to engage the stops comprises a rod portion of the handle.

13. A medical syringe comprising:
an outer housing having forward and rear end portions,
a syringe barrel of curvilinear cross-section slidable in the outer housing,
a resilient seal in a forward portion of the outer housing,
a hollow needle extending forwardly from the syringe barrel and movable therewith to penetrate the seal member and extend outwardly of the forward end of the outer housing,
spring means between the syringe barrel and a forward wall portion of the housing,
means to apply pressure on liquid in the syringe barrel and to apply partial vacuum to draw liquid into the syringe barrel via said hollow needle,
a plurality of spaced-apart stops defined on an upper portion of the outer housing,
a control handle pivotal relative to the syringe barrel, said handle having means to engage said stops for selective positioning of the syringe barrel and said needle thereon to extend the needle beyond the forward end portion of the outer housing, and to retract the needle within the outer housing end portion, and
a plunger manually movable to extend outwardly of the syringe barrel to draw liquid via said hollow needle from a container to the syringe barrel, the plunger being manually movable inwardly of the syringe barrel to exert pressure in the syringe barrel.

14. A syringe according to claim 13, wherein the control handle is biased outwardly of a pivot on the syringe barrel by spring means engaging the handle and the syringe barrel, the spring having arms engaging the handle and the syringe housing.

15. A syringe according to claim 13, wherein said syringe barrel is of generally cylindrical cross-section.

16. A syringe according to claim 13, wherein said means to apply pressure and partial vacuum comprises a plunger slidable in the syringe barrel.

17. A syringe according to claim 13, wherein the spring means comprises a coil spring.

18. A syringe according to claim 13, wherein a forward portion of the outer housing is of reduced cylindrical configuration and wherein said seal is mounted.

19. A medical syringe comprising:
an outer housing having forward and rear end portions,
a syringe barrel of curvilinear cross-section slidable in the outer housing,
a seal in a forward portion of the outer housing,
a hollow needle extending forwardly from the syringe barrel and movable therewith to penetrate the seal member and extend outwardly of the syringe body,
spring means between the syringe barrel and a forward wall portion of the housing,
a plurality of spaced-apart stops defined on an upper portion of the outer housing, and
a control handle movable relative to the syringe barrel, and means to engage any of the respective stops for the selective positioning of the needle to extend the needle beyond the forward end portion of the housing and to retract the needle within the housing end portion, manual pushing on the handle thus converting the syringe between a needle device or system and a needle-less device or system, the user presses down on the handle spring means to move the needle from its outward position rearwardly into the housing.

20. A syringe according to claim 19, wherein the control handle is biased outwardly of a pivot on the syringe barrel by spring means engaging the handle and the syringe barrel, the spring having respective arms engaging the handle and the outer housing.

21. A syringe according to claim 19, wherein said syringe barrel is of generally cylindrical cross-section.

22. A syringe according to claim 19, wherein the spring means comprises a coil spring.

23. A syringe according to claim 19, wherein a forward portion of the outer housing is of reduced cylindrical configuration and wherein said seal is mounted.

24. A syringe according to claim 19, wherein the plurality of stops on the upper housing to receive the handle means comprises spaced-apart notches in the upper wall of the outer housing, and the handle means to engage in the notches is a handle portion adapted to engage in the notches.

25. A syringe according to claim 19, wherein the means to engage the stops comprises a prong on the handle means.

26. A syringe according to claim 24, wherein the means to engage the stops comprises a rod portion of the handle.
